(19) United States
(12) Patent Application Publication
(10) Pub. No.: US 2025/0120694 A1
(43) Pub. Date: Apr. 17, 2025

(54) ADJUNCTS FOR USE WITH SURGICAL STAPLING INSTRUMENTS

(71) Applicant: Cilag GmbH International, Zug (CH)

(72) Inventors: Heather B. Strang, West Chester, OH (US); Jordan B. Wong, Cincinnati, OH (US); Frederick E. Shelton, IV, Hillsboro, OH (US); Michael J. Vendely, Lebanon, OH (US); Jason L. Harris, Lebanon, OH (US)

(21) Appl. No.: 18/379,822

(22) Filed: Oct. 13, 2023

(51) Int. Cl. A61B 17/068; A61B 17/072

(52) U.S. Cl. CPC A61B 17/0684; A61B 17/07292

(57) ABSTRACT

An adjunct for use with a surgical stapler is disclosed. The adjunct comprises a first layer comprising a first material. The adjunct comprises a second film layer (70070′) attached to the first layer. The second film layer defines a continuous textured device-contacting surface configured to establish a frictional interface with a corresponding portion of the surgical stapler. The second film layer comprises a second material that differs from the first material, the adjunct comprises a third film layer attached to the first layer. The third film layer comprises a tissue-contacting surface.

### BACKGROUND

The present invention relates to surgical instruments and, in various arrangements, to adjuncts, end effectors and staple cartridges for use with surgical stapling and cutting instruments that are designed to staple and cut tissue.

Adjunct materials are utilized in connection with surgical stapler end effectors and staple cartridges to provide various therapeutic advantages to the stapled tissue. Such materials are positioned between the cartridge and an anvil of the surgical stapler end effector and are stapled to the tissue as the tissue is cut and stapled. These adjunct materials are constructed to be absorbed by the body during the healing process. Depending upon the application, one adjunct may be situated adjacent the underside of the anvil and another adjunct may be positioned on the deck surface of the staple cartridge. The end effector is then advantageously manipulated to position the target tissue between the adjuncts and the jaws of the end effector are closed onto the target tissue. In other applications, only one adjunct is employed. In either case, after the jaws have been fully closed to clamp target tissue therebetween, the surgical stapler is fired. Firing entails the driving of a firing actuator or sled driver distally through the end effector that has a staple cartridge supported therein. Staples are stored on drivers that are movably supported in longitudinal rows within the staple cartridge. A sled is movably supported in the cartridge and is configured to sequentially cam the drivers in a direction toward the closed anvil. As each driver moves toward the closed anvil, the staple supported thereon is driven through the adjunct(s) and the clamped target tissue into forming contact with the underside of the anvil. The firing actuator or sled driver is also equipped with a knife or tissue cutting feature that severs the adjunct(s) and the clamped tissue. The knife is positioned to lag behind the sled so that the tissue is stapled before it is cut.

During the firing process, as the sled driver or firing actuator is driven distally, the knife must initially contact proximal end portion(s) of the adjunct(s) to cut through the adjunct materials. During this period of initial contact and before the staples are fired, the knife can cause the adjunct(s) to move and slip out of position, which can lead to malformed staples and mis-positioning of the adjunct(s) during stapling. This problem can be further acerbated when using adjuncts while sequentially firing multiple staple cartridges along a single tissue cut line. For example, when cutting and stapling a target tissue requiring multiple firings, a first staple cartridge is employed to cut and attach a first adjunct or adjuncts to a first portion of the target tissue. Thereafter, the spent staple cartridge is replaced with a fresh cartridge and a corresponding second adjunct or second adjuncts are supported in the end effector. The end effector jaws are positioned so that a proximal end of the second adjunct or adjuncts is overlapped with the distal end of the stapled first adjunct or adjuncts and then the jaws are closed to clamp the remaining portion of target tissue between the second adjunct(s) and the jaws. As the sled driver or firing actuator is driven distally, the knife on the firing actuator contacts the overlapping adjuncts. If the second adjunct(s) move during this process, they may bunch or “plow” which could cause the second adjuncts to detrimentally affect the staple formation process. This undesirable plowing or bunching is more likely to occur when the knife of the firing actuator or sled driver initially contacts the overlapping second adjunct(s). Thus, it is desirable for the adjuncts and end effectors to be configured to prevent the adjuncts from moving out of position during the cutting and staple firing procedures. The present disclosure provides for improvements and modifications which minimize and prevent adjunct slippage and movement during those operations.

### SUMMARY

An adjunct for use with a surgical stapler is disclosed. The adjunct comprises a first layer comprising a first material. The adjunct comprises a second film layer attached to the first layer. The second film layer defines a continuous textured device-contacting surface configured to establish a frictional interface with a corresponding portion of the surgical stapler. The second film layer comprises a second material that differs from the first material, the adjunct comprises a third film layer attached to the first layer. The third film layer comprises a tissue-contacting surface.

A surgical stapler end effector is disclosed. The surgical stapler end effector comprises a first jaw and an anvil. The anvil is movably supported relative to the first jaw. The anvil comprises a proximal end portion, a distal end portion, and a staple-forming undersurface extending from the proximal end portion to the distal end portion. The surgical stapler end effector comprises an adjunct removably coupled to the anvil adjacent the staple-forming undersurface. The adjunct comprises an adjunct retention assembly comprising at least one hook formed on a proximal end of the adjunct and is configured to releasably engage a corresponding hook portion of the proximal end of the anvil.

A surgical stapler end effector is disclosed. The surgical stapler end effector comprises a first jaw and an anvil. The anvil is movably supported relative to the first jaw between an open position and a closed position. The anvil comprises a proximal end portion, a distal end portion, an undersurface comprising a staple-forming portion, and at least one tissue stop on the proximal end, wherein each tissue stop defines a distal tissue stop edge. The anvil also comprises at least one projection protruding from a portion of the undersurface that is proximal to each of the distal tissue stop edges. Each projection is configured to pin a proximal end of an adjunct positioned adjacent the undersurface between the projection and a corresponding portion of the first jaw to prevent movement of the adjunct relative to the first jaw when the anvil is in the closed position.

### LISTING OF THE DRAWINGS

Various features of the embodiments described herein, together with advantages thereof, may be understood in accordance with the following description taken in conjunction with the accompanying drawings as follows:

FIG. 1 is a perspective view of a surgical stapler end effector;

FIG. 2 is a cross-sectional side view of an adjunct for use with a staple cartridge and/or anvil of a surgical stapler end effector in accordance with the present disclosure;

FIG. 3 is a side view of two of the adjuncts of FIG. 2 used in connection with an anvil and a staple cartridge of a surgical stapler end effector to clamp a portion of target tissue therebetween in accordance with the present disclosure;

FIG. 4 is a cross-sectional side view of another adjunct in accordance with the present disclosure;

FIG. 5 is a cross-sectional side view of another adjunct in accordance with the present disclosure;

FIG. 6 is a cross-sectional side view of another adjunct in accordance with the present disclosure;

FIG. 7 is a cross-sectional side view of another adjunct used in connection with a surgical staple cartridge of a surgical stapler end effector in accordance with the present disclosure;

FIG. 8 is a cross-sectional side view of another adjunct used in connection with a surgical staple cartridge of a surgical stapler end effector in accordance with the present disclosure;

FIG. 9 is a cross-sectional side view of another adjunct used in connection with an anvil of a surgical stapler end effector in accordance with the present disclosure;

FIG. 10 is a partial perspective view of a surgical stapler end effector in accordance with the present disclosure;

FIG. 11 is a cross-sectional end view of the surgical stapler end effector of FIG. 10 in accordance with the present disclosure;

FIG. 12 is partial cross-sectional side view of the surgical stapler end effector of FIG. 9 in accordance with the present disclosure;

FIG. 13 is another partial perspective view of the surgical stapler end effector of FIG. 9 with an adjunct supported on the staple cartridge of the end effector in accordance with the present disclosure;

FIG. 14 is a perspective view of an anvil and an adjunct of a surgical stapler end effector in accordance with the present disclosure;

FIG. 15 is a partial cross-sectional perspective view of proximal end portions of the anvil and adjunct of FIG. 14 in accordance with the present disclosure;

FIG. 16 is a top partial cross-sectional view of a portion of the adjunct and anvil of FIG. 14 used in connection with a surgical stapler end effector with the knife of the end effector in an initial position prior to commencement of a cutting and firing process in accordance with the present disclosure;

FIG. 17 is another top partial cross-sectional view of the adjunct, anvil and end effector of FIG. 16 with the knife in an intermediate position in accordance with the present disclosure; and

FIG. 18 is another top partial cross-sectional view of the adjunct, anvil, and end effector of FIGS. 16 and 17 showing an adjunct attachment assembly of the adjunct partially torn from the adjunct in accordance with the present disclosure.

### DETAILED DESCRIPTION

Applicant of the present application owns the following U.S. Patent Applications that were filed on even date herewith and which are each herein incorporated by reference in their respective entireties:
U.S. Patent Application, titled METHOD OF OPERATING A SURGICAL STAPLING INSTRUMENT; Attorney Docket No. END9484USNP2/220491-2M;
U.S. Patent Application, titled SURGICAL STAPLING SYSTEMS WITH ADAPTIVE STAPLE FIRING ALGORITHMS; Attorney Docket No. END9484USNP3/220491-3;
U.S. Patent Application, titled LEARNED TRIGGERS FOR ADAPTIVE CONTROL OF SURGICAL STAPLING SYSTEMS; Attorney Docket No. END9484USNP4/220491-4;
U.S. Patent Application, titled CONTROL CIRCUIT FOR ACTUATING MOTORIZED FUNCTION OF SURGICAL STAPLING INSTRUMENT UTILIZING INERTIAL DRIVE TRAIN PROPERTIES; Attorney Docket No. END9484USNP5/220491-5;
U.S. Patent Application, titled PROPORTIONATE BALANCING OF THE FUNCTION IMPACT MAGNITUDE OF BATTERY OUTPUT TO PEAK MOTOR CURRENT; Attorney Docket No. END9484USNP6/220491-6;
U.S. Patent Application, titled MOTOR OPTIMIZATION BY MINIMIZATION OF PARASITIC LOSSES AND TUNING MOTOR DRIVE CONFIGURATION; Attorney Docket No. END9484USNP7/220491-7;
U.S. Patent Application, titled APPARATUS AND METHOD TO REDUCE PARASITIC LOSSES OF THE ELECTRICAL SYSTEM OF A SURGICAL INSTRUMENT; Attorney Docket No. END9484USNP8/220491-8;
U.S. Patent Application, titled SURGICAL TOOL WITH RELAXED FLEX CIRCUIT ARTICULATION; Attorney Docket No. END9484USNP9/220491-9;
U.S. Patent Application, titled WIRING HARNESS FOR SMART STAPLER WITH MULTI AXIS ARTICULATION; Attorney Docket No. END9484USNP10/220491-10;
U.S. Patent Application, titled SURGICAL SYSTEM WITH WIRELESS ARRAY FOR POWER AND DATA TRANSFER; Attorney Docket No. END9484USNP11/220491-11; and.
U.S. Patent Application, titled SURGICAL STAPLE CARTRIDGE COMPRISING REPLACEABLE ELECTRONICS PACKAGE; Attorney Docket No. END9484USNP12/220491-12.

Applicant of the present application owns the following U.S. Patent Applications that were filed on even date herewith and which are each herein incorporated by reference in their respective entireties:
U.S. Patent Application, titled METHOD OF ASSEMBLING A STAPLE CARTRIDGE; Attorney Docket No. END9484USNP13/220491-13M;
U.S. Patent Application, titled CONTROL SURFACES ON A STAPLE DRIVER OF A SURGICAL STAPLE CARTRIDGE; Attorney Docket No. END9484USNP14/220491-14;
U.S. Patent Application, titled INTEGRAL CARTRIDGE STIFFENING FEATURES TO REDUCE CARTRIDGE DEFLECTION; Attorney Docket No. END9484USNP15/220491-15;
U.S. Patent Application, titled STAPLE CARTRIDGE COMPRISING WALL STRUCTURES TO REDUCE CARTRIDGE DEFLECTION; Attorney Docket No. END9484USNP16/220491-16;
U.S. Patent Application, titled PAN-LESS STAPLE CARTRIDGE ASSEMBLY COMPRISING RETENTION FEATURES FOR HOLDING STAPLE DRIVERS AND SLED; Attorney Docket No. END9484USNP17/220491-17;
U.S. Patent Application, titled STAPLE CARTRIDGE COMPRISING A SLED HAVING A DRIVER LIFT CAM; Attorney Docket No. END9484USNP18/220491-18;
U.S. Patent Application, titled SURGICAL STAPLE CARTRIDGES WITH SLEDS CONFIGURED TO BE COUPLED TO A FIRING DRIVER OF A COMPATIBLE SURGICAL STAPLER; Attorney Docket No. END9484USNP19/220491-19;
U.S. Patent Application, titled STAPLE CARTRIDGE COMPRISING A COMPOSITE SLED; Attorney Docket No. END9484USNP20/220491-20;
U.S. Patent Application, titled SURGICAL INSTRUMENTS WITH JAW AND FIRING ACTUATOR LOCKOUT ARRANGEMENTS LOCATED PROXIMAL TO A JAW PIVOT LOCATION; Attorney Docket No. END9484USNP21/220491-21;
U.S. Patent Application, titled SURGICAL INSTRUMENTS WITH LATERALLY ENGAGEABLE LOCKING ARRANGEMENTS FOR LOCKING A FIRING ACTUATOR; Attorney Docket No. END9484USNP22/220491-22
U.S. Patent Application, titled DUAL INDEPENDENT KEYED LOCKING MEMBERS ACTING ON THE SAME DRIVE MEMBER; Attorney Docket No. END9484USNP23/220491-23;
U.S. Patent Application, titled ADJUNCTS FOR USE WITH SURGICAL STAPLING INSTRUMENTS; Attorney Docket No. END9484USNP24/220491-24;
U.S. Patent Application, titled JAW CONTROL SURFACES ON A SURGICAL INSTRUMENT JAW; Attorney Docket No. END9484USNP26/220491-26
U.S. Patent Application, titled ZONED ALGORITHM ADAPTIVE CHANGES BASED ON CORRELATION OF COOPERATIVE COMPRESSION CONTRIBUTIONS OF TISSUE; Attorney Docket No. END9484USNP27/220491-27;
U.S. Patent Application, titled STAPLE CARTRIDGES COMPRISING TRACE RETENTION FEATURES; Attorney Docket No. END9484USNP29/220491-29; and. U.S. Patent Application, titled STAPLE CARTRIDGES COMPRISING STAPLE RETENTION FEATURES; Attorney Docket No. END9484USNP30/220491-30.

The entire disclosures of U.S. Pat. No. 11,589,865, entitled METHODS FOR CONTROLLING A POWERED SURGICAL STAPLER THAT HAS SEPARATE ROTARY CLOSURE AND FIRING SYSTEMS, which issued on Feb. 28, 2023, U.S. Pat. No. 6,978,921, entitled SURGICAL STAPLING INSTRUMENT INCORPORATING AN E-BEAM FIRING MECHANISM, which issued on Dec. 27, 2005, U.S. Pat. No. 10,213,203, entitled STAPLE CARTRIDGE ASSEMBLY WITHOUT A BOTTOM COVER, which issued on Feb. 26, 2019, U.S. Pat. No. 10,945,727, entitled STAPLE CARTRIDGE WITH DEFORMABLE DRIVER RETENTION FEATURES, which issued on Mar. 16, 2021, U.S. Pat. No. 11,234,698, entitled STAPLING SYSTEM COMPRISING A CLAMP LOCKOUT AND A FIRING LOCKOUT, which issued on Feb. 1, 2022, U.S. Pat. No. 11,540,826, entitled SURGICAL STAPLER END EFFECTOR SLED HAVING CARTRIDGE WALL SUPPORT FEATURE, which issued on Jan. 3, 2023, U.S. Pat. No. 10,299,792, entitled FASTENER CARTRIDGE COMPRISING NON-UNIFORM FASTENERS, which issued on May 28, 2019,U.S. Patent No. 8,540,133, entitled STAPLE CARTRIDGE, which issued on Sep. 24, 2013, U.S. Pat. No. 9,788,835, entitled DEVICES AND METHODS FOR FACILITATING EJECTION OF SURGICAL FASTENERS FROM CARTRIDGES, which issued on Oct. 17, 2017, U.S. Patent No. 10,105,142, entitled SURGICAL STAPLER WITH PLURALITY OF CUTTING ELEMENTS, which issued on Oct. 23, 2018, U.S. Pat. No. 10,537,324, entitled STEPPED STAPLE CARTRIDGE WITH ASYMMETRICAL STAPLES, which issued on Jan. 21, 2020, U.S. Pat. No. 7,669,746, entitled STAPLE CARTRIDGES FOR FORMING STAPLES HAVING DIFFERING FORMED STAPLE HEIGHTS, which issued on Mar. 2, 2010, U.S. Patent No. 8,123,100, entitled SURGICAL STAPLING INSTRUMENTS INCLUDING A CARTRIDGE HAVING MULTIPLE STAPLE SIZES, which issued on Feb. 28, 2012, U.S. Pat. No. 7,407,075, entitled STAPLE CARTRIDGE HAVING MULTIPLE STAPLE SIZES FOR A SURGICAL STAPLING INSTRUMENT, which issued on Aug. 5, 2008, U.S. Pat. No. 10,085,749, entitled SURGICAL APPARATUS WITH CONDUCTOR STRAIN RELIEF, which issued on Oct. 2, 2018, U.S. Pat. No. 10,765,427, entitled METHOD FOR ARTICULATING A SURGICAL INSTRUMENT, which issued on Sep. 8, 2020, U.S. Pat. No. 11,291,445, entitled SURGICAL STAPLE CARTRIDGES WITH INTEGRAL AUTHENTICATION KEYS, which issued on Apr. 5, 2022, U.S. Pat. No. 8,864,007, entitled IMPLANTABLE FASTENER CARTRIDGE HAVING A NON-UNIFORM ARRANGEMENT, which issued on Oct. 21, 2014, U.S. Pat. No. 11,490,890, entitled COMPRESSIBLE NON-FIBROUS ADJUNCTS, which issued on Nov. 8, 2022, U.S. Pat. No. 10,952,724, entitled THREE DIMENSIONAL ADJUNCTS, which issued on Mar. 23, 2021, U.S. Pat. No. 9,770,245, entitled LAYER ARRANGEMENTS FOR SURGICAL STAPLE CARTRIDGES, which issued on Sep. 26, 2017, U.S. Patent No. 10,123,798, entitled TISSUE THICKNESS COMPENSATOR COMPRISING CONTROLLED RELEASE AND EXPANSION, which issued on Nov. 13, 2018, U.S. Patent No. 10,166,023, entitled METHOD OF APPLYING A BUTTRESS TO A SURGICAL STAPLER END EFFECTOR, which issued on Jan. 1, 2019, U.S. Pat. No. 11,207,065, entitled METHOD FOR FABRICATING SURGICAL STAPLER ANVILS, which issued on Dec. 28, 2021, U.S. Patent No. 8,141,762, entitled SURGICAL STAPLER COMPRISING A STAPLE POCKET, which issued on Mar. 27, 2012, U.S. Pat. No. 8,876,857, entitled END EFFECTOR WITH REDUNDANT CLOSING MECHANISMS, which issued on Nov. 4, 2014, U.S. Pat. No. 9,629,631, entitled COMPOSITE DRIVE BEAM FOR SURGICAL STAPLING, which issued on Apr. 25, 2017, U.S. Patent Application Publication No. 2022/0346858, entitled METHOD FOR OPERATING A SURGICAL INSTRUMENT INCLUDING SEGMENTED ELECTRODES, which published on Nov. 3, 2022, U.S. Patent Application Publication No. 2022/0304680, entitled DRIVERS FOR FASTENER CARTRIDGE ASSEMBLIES HAVING ROTARY DRIVE SCREWS, which published on Sep. 29, 2022, U.S. Patent Application Publication No. 2022/0304679, entitled METHOD OF USING A POWERED STAPLING DEVICE, which published on Sep. 29, 2022, U.S. Patent Publication No. 2019/0298350, entitled METHODS FOR CONTROLLING A POWERED SURGICAL STAPLER THAT HAS SEPARATE ROTARY CLOSURE AND FIRING SYSTEMS, which published on Oct. 3, 2019, U.S. Patent Application Publication No. 2017/0367695, entitled STAPLE CARTRIDGE COMPRISING WIRE STAPLES AND STAMPED STAPLES, which published on Dec. 28, 2017, U.S. Patent Application Publication No. 2015/0134077, entitled SEALING MATERIALS FOR USE IN SURGICAL STAPLING, which published on May 14, 2015, U.S. Patent Application Publication No. 2018/0168615, entitled METHOD OF DEFORMING STAPLES FROM TWO DIFFERENT TYPES OF STAPLE CARTRIDGES WITH THE SAME SURGICAL STAPLING INSTRUMENT, which published on Jun. 21, 2018, U.S. Patent Application Publication No. 2018/0132849, entitled STAPLE FORMING POCKET CONFIGURATIONS FOR CIRCULAR SURGICAL STAPLER ANVIL, which published on May 17, 2018, U.S. Patent Application Publication No. 2018/0168613, entitled SURGICAL INSTRUMENTS WITH JAWS THAT ARE PIVOTABLE ABOUT A FIXED AXIS AND INCLUDE SEPARATE AND DISTINCT CLOSURE AND FIRING SYSTEMS, which published on Jun. 21, 2018, U.S. Patent Application Publication No. 2017/0319205, entitled POWERED END EFFECTOR ASSEMBLY WITH PIVOTABLE CHANNEL, which published on Nov. 9, 2017, U.S. Patent Application Publication No. 2014/0001231, entitled FIRING SYSTEM LOCKOUT ARRANGEMENTS FOR SURGICAL INSTRUMENTS, which published on Jan. 2, 2014, U.S. Patent Application Publication No. 2016/0095596, entitled APPARATUS FOR ENDOSCOPIC PROCEDURES, which published on Apr. 7, 2016, U.S. Patent Application Publication No. 2015/0297199, entitled ADAPTER ASSEMBLY WITH GIMBAL FOR INTERCONNECTING ELECTROMECHANICAL SURGICAL DEVICES AND SURGICAL LOADING UNITS, AND SURGICAL SYSTEMS THEREOF, which published on Oct. 22, 2015, U.S. Patent Application Publication No. 2022/0031351, entitled SURGICAL INSTRUMENTS WITH DIFFERENT ARTICULATION JOINT ARRANGEMENTS FOR ACCOMMODATING FLEXIBLE ACTUATORS, which published on Feb. 3, 2022, U.S. Patent Application Publication No. 2022/0031320, entitled SURGICAL INSTRUMENTS WITH FLEXIBLE FIRING MEMBER ACTUATOR CONSTRAINT ARRANGEMENTS, which published on Feb. 3, 2022, U.S. Patent Application Publication No. 2023/0119119, entitled CABLE-DRIVEN ACTUATION SYSTEM FOR ROBOTIC SURGICAL TOOL ATTACHMENT, which published on Apr. 20, 2023, International Patent Publication No. WO2018/071497, entitled STAPLER CARTRIDGE WITH AN INTEGRAL KNIFE, which published on Apr. 18, 2018, International Patent Publication No. WO2018/049211, entitled WRIST ARCHITECTURE, which published on Mar. 15, 2018, U.S. Patent No. 11,298, 129, entitled METHOD FOR PROVIDING AN AUTHENTICATION LOCKOUT IN A SURGICAL STAPLER WITH A REPLACEABLE CARTRIDGE, which issued on Apr. 12, 2022, U.S. Patent No. 10,898, 183, entitled ROBOTIC SURGICAL INSTRUMENT WITH CLOSED LOOP FEEDBACK TECHNIQUES FOR ADVANCEMENT OF CLOSURE MEMBER DURING FIRING, which issued on Jan. 26, 2021, U.S. Pat. No. 5,485,947, entitled LINEAR STAPLING MECHANISM WITH CUTTING MEANS, which issued on Jan. 23, 1996, International Patent Publication No. WO2018/049206, entitled STAPLER RELOAD DETECTION AND IDENTIFICATION, which published on Mar. 15, 2018, U.S. Patent Application Publication No. 2016/0249920, entitled Surgical fastener applying apparatus, which published on Sep. 1, 2016, U.S. Design Patent No. D974,560, entitled STAPLE CARTRIDGE, which issued on Jan. 3, 2023, U.S. Design Patent No. D967,421, entitled STAPLE CARTRIDGE, which issued on Oct. 18, 2022, U.S. Design Patent No. D933,220, entitled BUTTRESS ASSEMBLY FOR A SURGICAL STAPLER, which issued on Oct. 12, 2021, U.S. Pat. No. 9,839,420, entitled TISSUE THICKNESS COMPENSATOR COMPRISING AT LEAST ONE MEDICAMENT, which issued on Dec. 12, 2017, U.S. Pat. No. 10,588,623, entitled ADHESIVE FILM LAMINATE, which issued on Mar. 17, 2020, U.S. Pat. No. 8,499,992, entitled DEVICE AND METHOD FOR CONTROLLING COMPRESSION OF TISSUE, which issued on Aug. 6, 2013, U.S. Patent Application Publication No. 2022/0378427, entitled STAPLING INSTRUMENT COMPRISING JAW MOUNTS, which published on Dec. 1, 2022, U.S. Pat. No. 10,349,939, entitled METHOD OF APPLYING A BUTTRESS TO A SURGICAL STAPLER, which issued on Jul. 16, 2019, U.S. Pat. No. 9,386,988, entitled RETAINER ASSEMBLY INCLUDING A TISSUE THICKNESS COMPENSATOR, which issued on Jul. 12, 2016, U.S. Pat. No. 9,072,535, entitled SURGICAL STAPLING INSTRUMENTS WITH ROTATABLE STAPLE DEPLOYMENT ARRANGEMENTS, which issued on Jul. 7, 2015, and U.S. Pat. No. 9,844,369, entitled, SURGICAL END EFFECTORS WITH FIRING ELEMENT MONITORING ARRANGEMENTS, which issued on Dec. 19, 2017 are incorporated by reference herein.

Numerous specific details are set forth to provide a thorough understanding of the overall structure, function, manufacture, and use of the embodiments as described in the specification and illustrated in the accompanying drawings. Well-known operations, components, and elements have not been described in detail so as not to obscure the embodiments described in the specification. The reader will understand that the embodiments described and illustrated herein are non-limiting examples, and thus it can be appreciated that the specific structural and functional details disclosed herein may be representative and illustrative. Variations and changes thereto may be made without departing from the scope of the claims.

The terms “proximal” and “distal” are used herein with reference to a clinician manipulating the handle portion of the surgical instrument. The term “proximal” refers to the portion closest to the clinician and the term “distal” refers to the portion located away from the clinician. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

Various exemplary devices and methods are provided for performing laparoscopic and minimally invasive surgical procedures. However, the reader will readily appreciate that the various methods and devices disclosed herein can be used in numerous surgical procedures and applications including, for example, in connection with open surgical procedures. As the present Detailed Description proceeds, the reader will further appreciate that the various instruments disclosed herein can be inserted into a body in any way, such as through a natural orifice, through an incision or puncture hole formed in tissue, etc. The working portions or end effector portions of the instruments can be inserted directly into a patient's body or can be inserted through an access device that has a working channel through which the end effector and elongate shaft of a surgical instrument can be advanced.

A surgical stapling system can comprise a shaft and an end effector extending from the shaft. The end effector comprises a first jaw and a second jaw. The first jaw comprises a staple cartridge. The staple cartridge is insertable into and removable from the first jaw; however, a staple cartridge may not be removable from, or at least readily replaceable from, the first jaw. The second jaw comprises an anvil configured to deform staples ejected from the staple cartridge. The second jaw is pivotable relative to the first jaw about a closure axis; however, the first jaw may be pivotable relative to the second jaw. The surgical stapling system further comprises an articulation joint configured to permit the end effector to be rotated, or articulated, relative to the shaft. The end effector is rotatable about an articulation axis extending through the articulation joint. Alternatively, the surgical stapling system may not include an articulation joint.

The staple cartridge comprises a cartridge body. The cartridge body includes a proximal end, a distal end, and a deck extending between the proximal end and the distal end. In use, the staple cartridge is positioned on a first side of the tissue to be stapled and the anvil is positioned on a second side of the tissue. The anvil is moved toward the staple cartridge to compress and clamp the tissue against the deck. Thereafter, staples removably stored in the cartridge body can be deployed into the tissue. The cartridge body includes staple cavities defined therein wherein staples are removably stored in the staple cavities. The staple cavities are arranged in six longitudinal rows. Three rows of staple cavities are positioned on a first side of a longitudinal slot and three rows of staple cavities are positioned on a second side of the longitudinal slot. Other arrangements of staple cavities and staples may be possible.

The staples are supported by staple drivers in the cartridge body. The drivers are movable between a first, or unfired position, and a second, or fired, position to eject the staples from the staple cavities. The drivers are retained in the cartridge body by a retainer which extends around the bottom of the cartridge body and includes resilient members configured to grip the cartridge body and hold the retainer to the cartridge body. The drivers are movable between their unfired positions and their fired positions by a sled. The sled is movable between a proximal position adjacent the proximal end and a distal position adjacent the distal end. The sled comprises a plurality of ramped surfaces configured to slide under the drivers and lift the drivers, and the staples supported thereon, toward the anvil.

Further to the above, the sled is moved distally by a firing driver. The firing driver is configured to contact the sled and push the sled toward the distal end. The longitudinal slot defined in the cartridge body is configured to receive the firing driver. The anvil also includes a slot configured to receive the firing driver. The firing driver further comprises a first cam which engages the first jaw and a second cam which engages the second jaw. As the firing driver is advanced distally, the first cam and the second cam can control the distance, or tissue gap, between the deck of the staple cartridge and the anvil. The firing driver also comprises a knife configured to incise the tissue captured intermediate the staple cartridge and the anvil. It is desirable for the knife to be positioned at least partially proximal to the ramped surfaces such that the staples are ejected ahead of the knife.

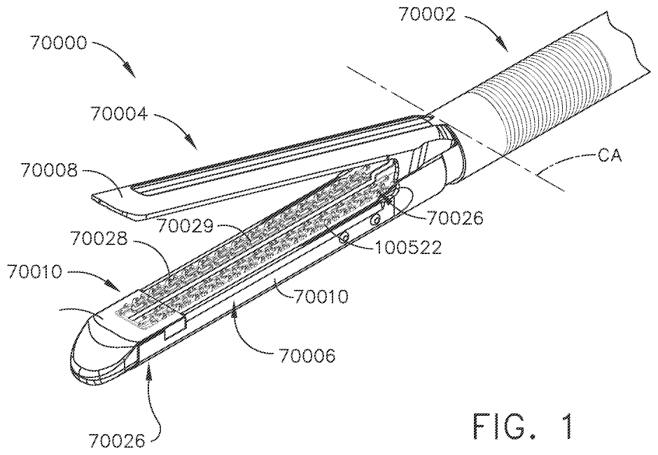

FIG. 1 depicts a surgical stapler 70000 comprising a shaft 70002 that includes an end effector 70004 that extends from the shaft 70002. The end effector 70004 comprises a first jaw 70006 and a second jaw 70008. The first jaw 70006 comprises an elongate channel 70010. A staple cartridge 70020 is insertable into and removable from the elongate channel 70010. However, the staple cartridge 70020 may not be removable from, or at least readily replaceable from, the first jaw 70006. The second jaw 70008 comprises an anvil 70030 that is configured to form staples that are ejected from the staple cartridge 70020. The second jaw 70008 is pivotable relative to the first jaw 70006 between an open position and a closed position; however, the first jaw 70006 may be pivotable relative to the second jaw 70008.

The staple cartridge 70020 includes a cartridge body 70022 that comprises a proximal end 70024 and a distal end 70026. The cartridge body 70022 further comprises a deck surface 70028 that extends between the proximal end 70024 and the distal end 70026 and is configured to oppose the anvil 70030. A longitudinal slot 70029 extends from the proximal end 70024 toward the distal end 70026 of the cartridge body 70022 and is sized to receive a sled driver or firing actuator that is configured to eject staples out of the cartridge body 70022 during a cutting and staple firing stroke. Various aspects of staple cartridges are described in greater detail in U.S. Pat. No. 9,844,369, the disclosure of which is herein incorporated by reference in its entirety.

Many surgical applications require the use of buttresses or tissue thickness compensators that are commonly referred to as adjuncts to be used in conjunction with the stapling end effectors described herein in order to reinforce a staple line and provide support to the tissue surrounding the staple line. Various adjuncts are disclosed herein and may comprise materials characterized by one or more of the following properties: biocompatible, bioabsorable, bioresorbable, biodurable, biodegradable, compressible, fluid absorbable, swellable, self-expandable, bioactive, medicament, pharmaceutically active, anti-adhesion, haemostatic, antibiotic, anti-microbial, anti-viral, nutritional, adhesive, permeable, hydrophilic and/or hydrophobic, for example. Any of the adjuncts disclosed herein may comprise at least one of a haemostatic agent, such as fibrin and thrombin, an antibiotic, such as doxycpl, and medicament, such as matrix metalloproteinases (MMPs).

The various adjuncts disclosed herein may comprise synthetic and/or non-synthetic materials. An adjunct may comprise a polymeric composition comprising one or more synthetic polymers and/or one or more non-synthetic polymers. The synthetic polymer may comprise a synthetic absorbable polymer and/or a synthetic non-absorbable polymer. The polymeric composition may comprise a biocompatible foam, for example. The biocompatible foam may comprise a porous, open cell foam and/or a porous, closed cell foam, for example. The biocompatible foam may have a uniform pore morphology or may have a gradient pore morphology (i.e. small pores gradually increasing in size to large pores across the thickness of the foam in one direction). The polymeric composition may comprise one or more of a porous scaffold, a porous matrix, a gel matrix, a hydrogel matrix, a solution matrix, a filamentous matrix, a tubular matrix, a composite matrix, a membranous matrix, a biostable polymer, and a biodegradable polymer, and combinations thereof. For example, the adjunct may comprise a foam reinforced by a filamentous matrix or may comprise a foam having an additional hydrogel layer that expands in the presence of bodily fluids to further provide the compression on the tissue. Any of the adjuncts disclosed herein could also be comprised of a coating on a material and/or a second or third layer that expands in the presence of bodily fluids to further provide the compression on the tissue. Such a layer could be a hydrogel that could be a synthetic and/or naturally derived material and could be either biodurable and/or biodegradable, for example. The adjunct may comprise a microgel or a nanogel. The hydrogel may comprise carbohydrate-derived microgels and/or nanogels. An adjunct may be reinforced with fibrous non-woven materials or fibrous mesh type elements, for example, that can provide additional flexibility, stiffness, and/or strength. An adjunct may have a porous morphology which exhibits a gradient structure such as, for example, small pores on one surface and larger pores on the other surface. Such morphology could be more optimal for tissue in-growth or haemostatic behavior. Further, the gradient could be also compositional with a varying bio-absorption profile. A short-term absorption profile may be preferred to address hemostasis while a long-term absorption profile may address better tissue healing without leakages.

Examples of non-synthetic materials from which an adjunct disclosed herein may be comprised of include, but are not limited to, lyophilized polysaccharide, glycoprotein, bovine pericardium, collagen, gelatin, fibrin, fibrinogen, elastin, proteoglycan, keratin, albumin, hydroxyethyl cellulose, cellulose, oxidized cellulose, oxidized regenerated cellulose (ORC), hydroxypropyl cellulose, carboxyethyl cellulose, carboxymethylcellulose, chitan, chitosan, casein, alginate, and combinations thereof.

Examples of synthetic absorbable materials include, but are not limited to, poly(lactic acid) (PLA), poly(L-lactic acid) (PLLA), polycaprolactone (PCL), polyglycolic acid (PGA), poly(trimethylene carbonate) (TMC), polyethylene terephthalate (PET), polyhydroxyalkanoate (PHA), a copolymer of glycolide and ε-caprolactone (PGCL), a copolymer of glycolide and -trimethylene carbonate, poly(glycerol sebacate) (PGS), poly(dioxanone) (PDS), polyesters, poly(orthoesters), polyoxaesters, polyetheresters, polycarbonates, polyamide esters, polyanhydrides, polysaccharides, poly(ester-amides), tyrosine-based polyarylates, polyamines, tyrosine-based polyiminocarbonates, tyrosine-based polycarbonates, poly(D,L-lactide-urethane), poly(hydroxybutyrate), poly(B-hydroxybutyrate), poly(E-caprolactone), polyethyleneglycol (PEG), poly [bis(carboxylatophenoxy) phosphazene]poly(amino acids), pseudo-poly(amino acids), absorbable polyurethanes, poly(phosphazine), polyphosphazenes, polyalkyleneoxides, polyacrylamides, polyhydroxyethylmethylacrylate, polyvinylpyrrolidone, polyvinyl alcohols, poly(caprolactone), polyacrylic acid, polyacetate, polypropylene, aliphatic polyesters, glycerols, copoly(ether-esters), polyalkylene oxalates, polyamides, poly(iminocarbonates), polyalkylene oxalates, and combinations thereof. The polyester is may be selected from the group consisting of polylactides, polyglycolides, trimethylene carbonates, polydioxanones, polycaprolactones, polybutesters, and combinations thereof.

The adjunct may comprise a surfactant. Examples of surfactants may include, but are not limited to, polyacrylic acid, methalose, methyl cellulose, ethyl cellulose, propyl cellulose, hydroxy ethyl cellulose, carboxy methyl cellulose, polyoxyethylene cetyl ether, polyoxyethylene lauryl ether, polyoxyethylene octyl ether, polyoxyethylene octylphenyl ether, polyoxyethylene oleyl ether, polyoxyethylene sorbitan monolaurate, polyoxyethylene stearyl ether, polyoxyethylene nonylphenyl ether, dialkylphenoxy poly(ethyleneoxy) ethanol, and polyoxamers.

The polymeric composition may comprise a pharmaceutically active agent. The polymeric composition may release a therapeutically effective amount of the pharmaceutically active agent. The pharmaceutically active agent may be released as the polymeric composition is desorbed/absorbed. The pharmaceutically active agent may be released into fluid, such as, for example, blood, passing over or through the polymeric composition. Examples of pharmaceutically active agents may include, but are not limited to, haemostatic agents and drugs, such as, for example, fibrin, thrombin, and oxidized regenerated cellulose (ORC); anti-inflammatory drugs, such as, for example, diclofenac, aspirin, naproxen, sulindac, and hydrocortisone; antibiotic and antimicrobial drug or agents, such as, for example, triclosan, ionic silver, ampicillin, gentamicin, polymyxin B, chloramphenicol; and anticancer agents, such as, for example, cisplatin, mitomycin, adriamycin.

The polymeric composition may comprise a haemostatic material. The adjunct may comprise haemostatic materials comprising poly(lactic acid), poly(glycolic acid), poly(hydroxybutyrate), poly(caprolactone), poly(dioxanone), polyalkyleneoxides, copoly(ether-esters), collagen, gelatin, thrombin, fibrin, fibrinogen, fibronectin, elastin, albumin, hemoglobin, ovalbumin, polysaccharides, hyaluronic acid, chondroitin sulfate, hydroxyethyl starch, hydroxyethyl cellulose, cellulose, oxidized cellulose, hydroxypropyl cellulose, carboxyethyl cellulose, carboxymethyl cellulose, chitan, chitosan, agarose, maltose, maltodextrin, alginate, clotting factors, methacrylate, polyurethanes, cyanoacrylates, platelet agonists, vasoconstrictors, alum, calcium, RGD peptides, proteins, protamine sulfate, ε-amino caproic acid, ferric sulfate, ferric subsulfates, ferric chloride, zinc, zinc chloride, aluminum chloride, aluminum sulfates, aluminum acetates, permanganates, tannins, bone wax, polyethylene glycols, fucans and combinations thereof. The adjunct may be characterized by haemostatic properties.

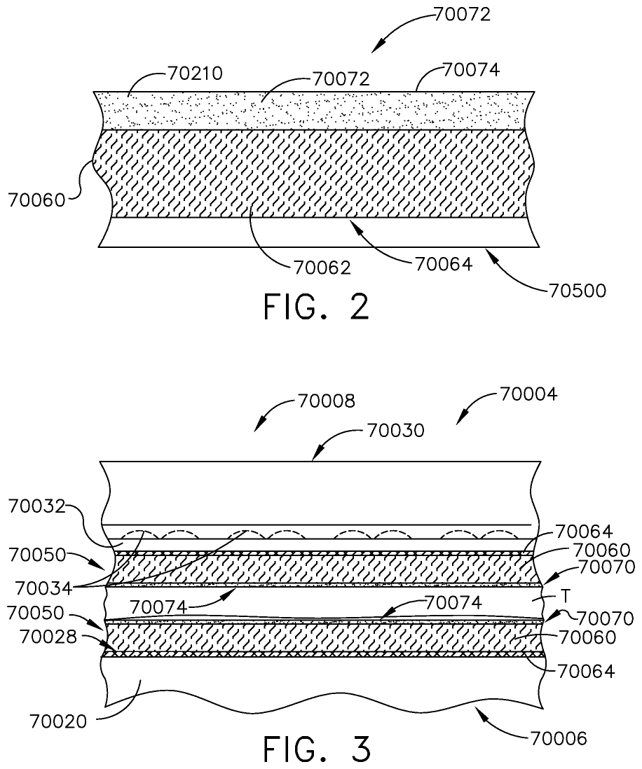

FIG. 2 illustrates an adjunct 70050 in accordance with the present disclosure. The adjunct 70050 comprises a first material layer 70060 that may comprises a Vicryl® mesh material 70062. The Vicryl® mesh material provides a woven-type of support structure for supporting the surgical staples as well as the severed tissue when fastened thereto. The first material layer 70060 may alternatively comprise other mesh or woven materials described herein. In the illustrated arrangement, the first material layer 70060 defines a first device-contacting surface 70064. The adjunct 70050 further comprises a second film layer 70070 that is adhered to a side of the first material layer 70060 that is opposite to the device-contacting surface 70064. The second film layer 70070 can be formed from a second material 70072 that differs from the first material 70062 and comprises a polydioxanone (PDO) film that helps to further stabilize the first mesh material 70062. In the illustrated arrangement, the second film layer 70070 defines a planar or substantially planar tissue-contacting surface 70074. The tissue-contacting surface 70074 is smooth as compared to the mesh, woven or lattice device-contacting surface 70064. Alternatively, the second film layer 70070 may be formed from alternative synthetic polymer materials described herein.

As can be seen in FIG. 3, in use, the adjunct 70050 may be positioned between the jaws 70006, 70008 of a surgical stapler end effector 70004 that comprises a surgical staple cartridge 70020 and an anvil 70030, respectively. In applications wherein only one adjunct 70050 is employed, the device-contacting surface 70064 is either placed in contact with a staple-forming undersurface 70032 of the anvil 70030 or a deck surface 70028 on the staple cartridge 70020. Those of ordinary skill in the art will appreciate that the mesh, woven or lattice configuration of the device-contacting surface 70064 will serve to establish a frictional interface with the staple-forming undersurface 70032 of the anvil 70030 or the deck surface 70028 of the staple cartridge 70020 to prevent the adjunct 70050 from slipping or otherwise moving laterally and/or longitudinally when initially contacted by the knife of the firing actuator or sled driver of the surgical stapler. FIG. 3 illustrates an application wherein two adjuncts 70050 are employed. As can be seen in FIG. 3, when two adjuncts 70050 are employed, the device-contacting surface 70064 of one adjunct 70050 is placed in contact with the staple-forming undersurface 70032 of the anvil 70030 and the device-contacting surface 70064 of the other adjunct 70050 is placed in contact with the deck surface 70028 of the staple cartridge 70020. The target tissue T is then clamped between the tissue-contacting surfaces 70074 of each of the adjuncts 70050.

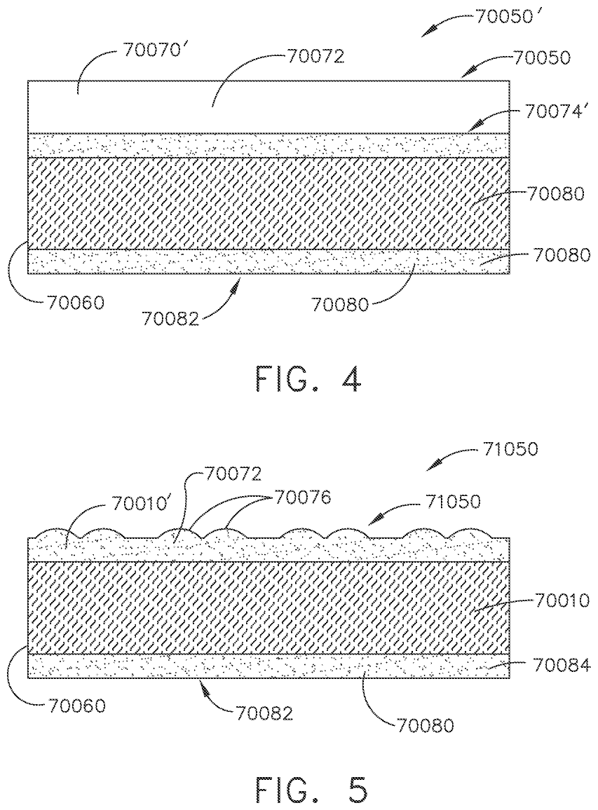

FIG. 4 depicts another adjunct 70050′ that is somewhat similar to adjunct 70050. As can be seen in FIG. 4, adjunct 70050′ additionally comprises a third film layer 70080 that defines a tissue-contacting surface 70082. The third film layer comprises a third material 70084 which, can be the same as the second material 70072. That is, the third material 70084 also comprises polydioxanone (PDO) film. The first film layer 70060 and the third film layer 70080 may be applied to the second material layer in one operation such that the second material layer is completely encapsulated in the polydioxanone (PDO) film. As such, the second film layer 70070′ defines a planar or substantially planar textured or roughened device-contacting surface 70074′.

In use, the adjunct 70050′ may be positioned between the jaws of a surgical stapler end effector that comprises an anvil and a surgical staple cartridge. In applications wherein only one adjunct 70050′ is employed, the device-contacting surface 70074′ is either placed in contact with the staple-forming undersurface of the anvil or the deck surface of the staple cartridge. Those of ordinary skill in the art will appreciate that the textured or roughened surface 70074′ will serve to establish a frictional interface with the anvil or staple cartridge to prevent the adjunct 70050′ from slipping or moving when initially contacted by the knife of the firing actuator or sled driver of the surgical stapler. In applications wherein two adjuncts 70050′ are employed, the device-contacting surface 70074′ of one adjunct 70050′ is placed in contact with the staple-forming surface of the anvil and the device-contacting surface 70074′ of the other adjunct 70050′ is placed in contact with the deck surface of the staple cartridge. The target tissue is then clamped between the tissue-contacting surfaces 70074′ of each of the adjuncts 70050′. The layered adjunct 70050′ enjoys the advantages provided by the woven material 70062 as well as the advantages provided by the non-woven material(s) 70072, 70084 while also minimizing the likelihood of the adjunct 70050′ moving, plowing or bunching during cutting and firing.

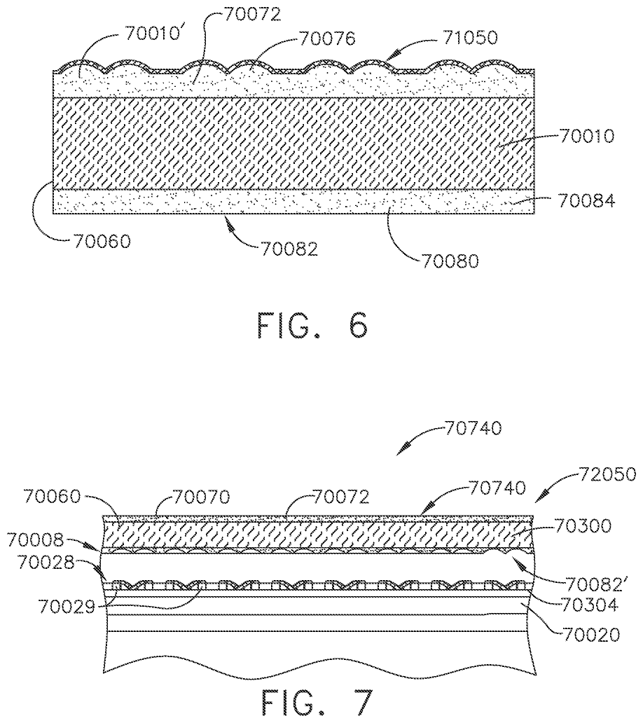

FIG. 5 illustrates another adjunct 71050 that is similar to the adjunct 70050′ described above, except that the device-contacting surface 71074 of the second film layer 70070′ is contoured to frictionally engage the staple-forming undersurface 70032 of the anvil 70030. For example, the device-contacting surface 71074 comprises a plurality of convex friction bumps 71076 that correspond to the staple-forming pockets 70034 in the anvil 70030. In use, the adjunct 71050 is placed adjacent to the anvil so that the friction bumps 71076 frictionally engage corresponding staple-forming pockets 70034 in the anvil 70030. Such arrangement serves to prevent the adjunct 71050 from slipping or moving when initially contacted by the knife of the firing actuator or sled driver of the surgical stapler. Alternatively, the contoured device-contacting surface may also comprise a continuous roughened or textured surface 70078 to further enhance the frictional engagement between the adjunct 71050 and the anvil 70030. See FIG. 6.

FIG. 7 depicts an adjunct 72050 that is similar to adjunct 70050′ except that the device-contacting surface 70082′ of the third film layer 70080′ is contoured to frictionally engage the deck 70028 of the surgical staple cartridge 70020. As can be seen in FIG. 7, the deck surface 70028 of the staple cartridge 70020 additionally comprises a plurality of features or protrusions 70029 that correspond to the staple pockets in the staple cartridge 70020. In the illustrated arrangement, the device-contacting surface 70082′ has a plurality of concave cavities 70086 that correspond to the protrusions 70029 on the staple cartridge 70020. Such arrangement serves to prevent the adjunct 72050 from slipping or moving when initially contacted by the knife of the firing actuator or sled driver of the surgical stapler. Alternatively, the contoured device-contacting surface 70082′ may also comprise a roughened or textured surface to further enhance the frictional engagement between the adjunct 72050 and the deck 70028 of the staple cartridge 70020.

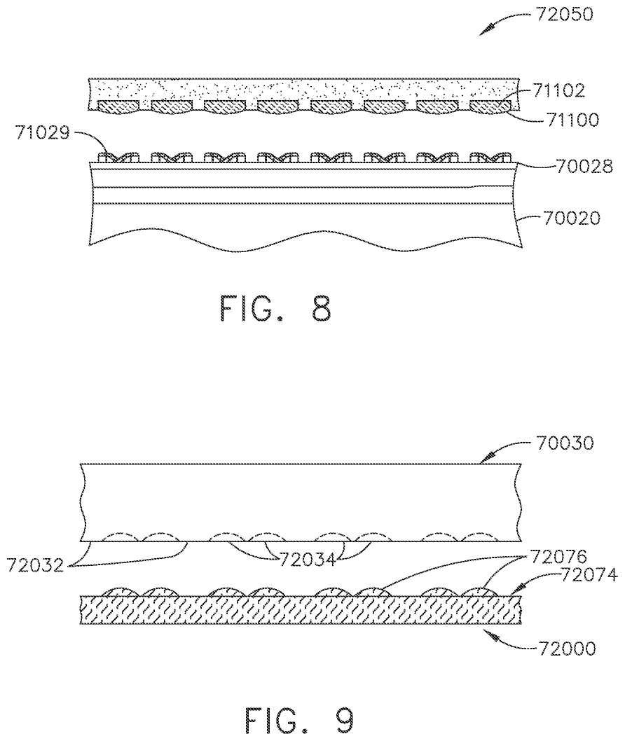

An adjunct 72050′ and a staple cartridge 70020 are illustrated in FIG. 8. The staple cartridge 70020 comprises a cartridge deck 70028 and cartridge deck features 70029. The adjunct 72050′ comprises a layer of first material 72060 that contains a pattern of traction bumps 71100. The first material 72060 may comprise any of the various adjunct materials disclosed herein. The first layer may comprise polydioxanone (PDO) or other synthetic polymer material disclosed herein. The traction bumps 71100 are fabricated from a second material 71102 that is embedded into the first material 72060 and has a coefficient of friction that is greater than the coefficient of friction of the first material 72060. For example, the second material may comprise a Vicryl® mesh. The traction bumps 71100 are configured to contact the cartridge deck features 71220. The traction bumps 71100 of the adjunct 71000 are configured to reduce slipping or sliding of the adjunct 72050′ relative to the staple cartridge 70020 and staple cartridge deck 70028.

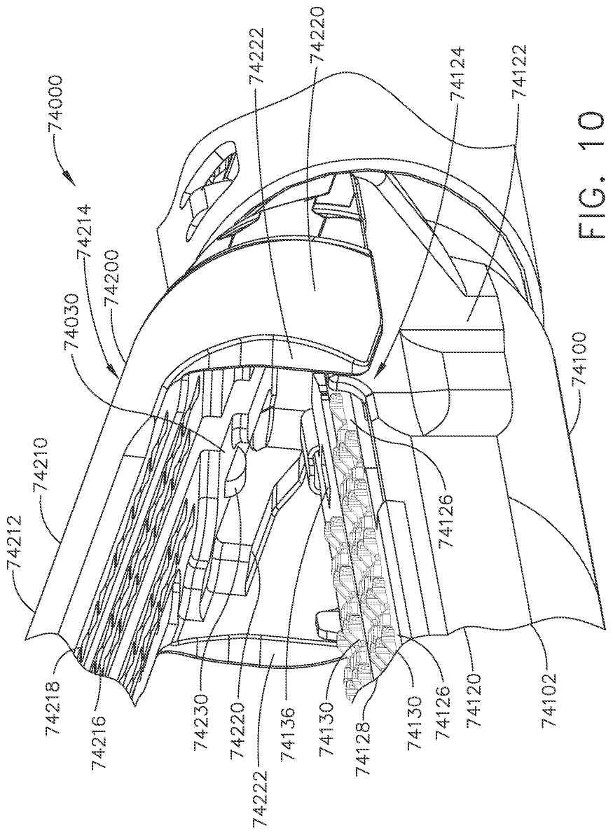

FIG. 9 illustrates another adjunct 72000 is formed from a single material 72002. For example, the material 72002 may comprise polydioxanone (PDO) or other synthetic polymer material disclosed herein. The adjunct 72000 defines a device-contacting surface 72074 comprises a plurality of convex friction bumps 72076 that correspond to the staple-forming pockets 70034 in the anvil 70030. In use, the adjunct 72000 is placed adjacent to the anvil 70030 so that the friction bumps 72076 frictionally engage corresponding staple-forming pockets 70034 in the anvil 70030. Such arrangement serves to prevent the adjunct 72000 from slipping or moving when initially contacted by the knife of the firing actuator or sled driver of the surgical stapler. Alternatively, the contoured device-contacting surface may also comprise a roughened or textured surface to further increase the frictional engagement between the adjunct 72000 and the anvil 70030.

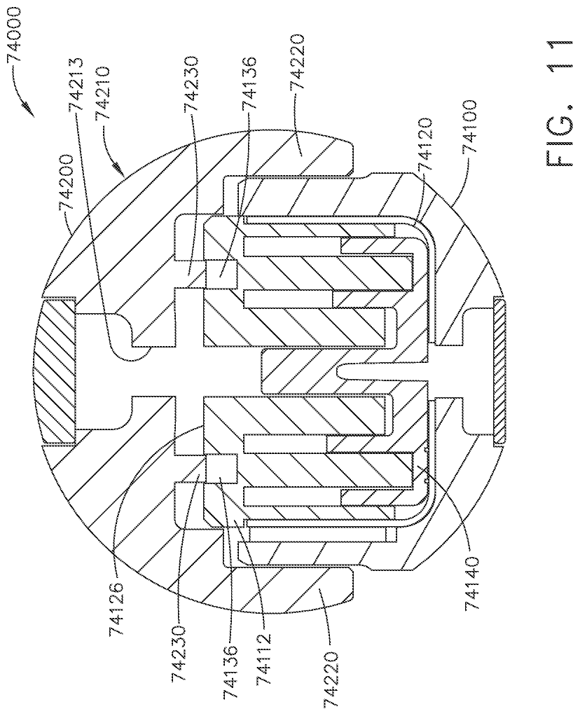

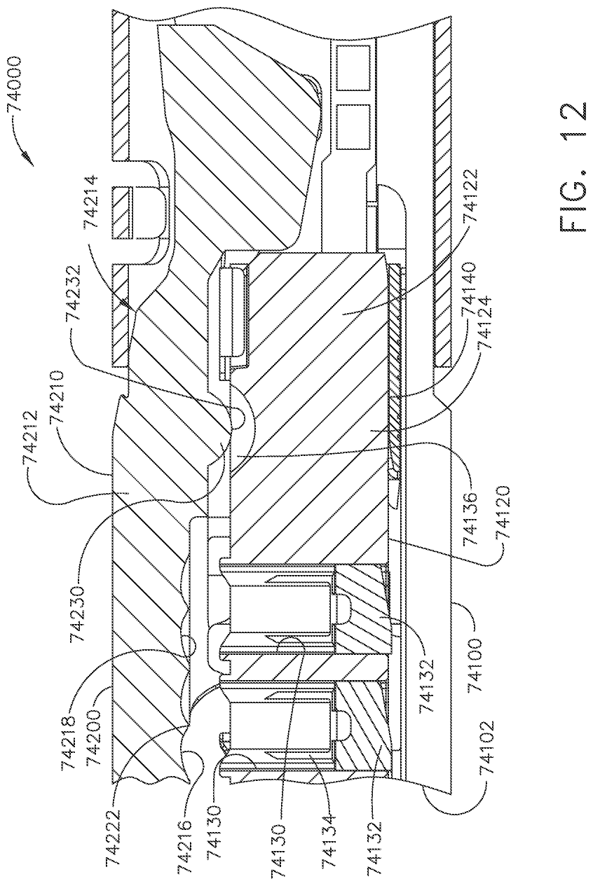

A surgical stapler end effector 74000 in accordance with the present disclosure is illustrated in FIGS. 10-13. The end effector 74000 comprises a first jaw 74100 and a second jaw 74200. The first jaw 74100 comprises an elongate channel 74102 that is configured to support a surgical staple cartridge 74120 therein. The staple cartridge 74120 is insertable into and removable from the elongate channel 74102; however, the staple cartridge 74120 may not be removable from, or at least readily replaceable from, the elongate channel 74102. The staple cartridge 74120 comprises a cartridge body 74122 that has a proximal end portion 74124 and a distal end portion (not shown). The cartridge body 74122 defines a deck surface 74126 that extends from the proximal end portion 74124 to the distal end portion of the cartridge body 74122. An elongate slot 74128 extends from the proximal end portion 74214 to the distal end portion to accommodate passage of a sled 74140 (and sled driver or firing actuator of the surgical stapling instrument) therethrough. See FIG. 11. Three longitudinal lines of lines staple cavities 74130 can be provided on each side of the elongate slot 74128. A corresponding staple driver 74132 is movably supported in each staple cavity 74130. One or more surgical staples 74134 or fasteners are removably supported on each driver 74132. See FIG. 12. Other staple cartridge arrangements and fastener arrangements are contemplated wherein the each fastener has an integral driver formed therewith. In any event, as the sled 74140 is driven distally by a sled driver or firing actuator of the surgical stapling instrument, the sled 74140 sequentially contacts the drivers 74132 in each longitudinal row and drives the drivers and the staples 74134 or fasteners supported thereon out of the corresponding staple cavity.

The second jaw 74200 comprises an anvil 74210 which is configured to deform staples 74134 ejected from the staple cartridge 74120. The anvil 74210 comprises an anvil body 74212 that has a proximal end portion 74214 and a distal end portion (not shown). A staple-forming surface 74216 extends from the proximal end portion 74214 to the distal end portion and defines a plurality of staple-forming pockets 74218 therein. An elongate anvil slot 74213 extends from the proximal end portion 74214 to the distal end portion to accommodate passage of the sled driver or firing actuator. The anvil 74210 further comprises a pair of tissue stops 74220 that protrude downward on each side of the proximal end portion 74214. The tissue stops 74220 each define a distal edge 74222 that is configured to be contacted by the target tissue when the target tissue is positioned between the anvil and the staple cartridge to prevent the target tissue from being positioned proximally beyond the proximal-most staples in the staple cartridge 74120.

The anvil 74210 further comprises at least one adjunct retainer in the form of a protrusion, tooth, or dimple 74230 that is formed on the proximal end portion 74214 and protrudes downwardly toward the staple cartridge 74120. As can be seen in FIG. 11, one protrusion 74230 is formed on one side of the anvil slot 74213 and another protrusion 74230 is formed on the other side of the anvil slot 74213. As can be seen in FIG. 12, each protrusion 74230 comprises a rounded surface 74232 that corresponds with a concave cavity, aperture or hole 74136 provided in the proximal end 74124 of the staple cartridge 74120. The rounded surface 74232 reduces the chance of tissue damage or trauma should tissue be inadvertently captured between the proximal end portion 74214 of the anvil 74210 and the proximal end 74122 of the staple cartridge 74120. However, as can be seen in FIG. 12, the protrusions 74230 can be proximal to the distal edges 74222 of the tissue stops 74220 as well as proximal to the proximal-most staple cavities 74130 and staples 74132 in the staple cartridge 74120. Thus, the tissue stops 74120 will prevent tissue from inadvertently migrating proximally to a point wherein the tissue could be inadvertently captured or clamped between the protrusions 74230 and their corresponding cavities 74136 in the staple cartridge 74120 as the anvil 74210 is closed.

FIG. 13 illustrates use of the end effector 74000 with an adjunct 74300. The adjunct 74300 may comprise any of the adjuncts disclosed herein. The adjunct 74300 may comprise a single layer of any of the adjunct materials disclosed herein, woven or non-woven. In one application, the adjunct 74300 is positioned on the deck surface 74126 of the staple cartridge 74120 such that a proximal end 74302 of the adjunct extends proximally beyond the cavities 74136 in the proximal end 74124 of the staple cartridge 74120. Thereafter, the end effector 74000 may be manipulated to position the target tissue between the staple-forming surface 74216 of the anvil 74210 and the adjunct 74300. Once the target tissue has been properly positioned, the anvil 74210 is closed to clamp the target tissue between the anvil and the adjunct 74300. As the anvil is closed, the protrusions 74230 clamp portions of the adjunct 74300 in their respective cavities 74136 to retain the adjunct 74300 in position and prevent any movement of the adjunct 74300 (laterally or longitudinally) when the knife of the sled driver or firing actuator initially contacts and cuts through the adjunct 74300. Alternatively, the adjunct 74300 can additionally comprise cavities, detents, apertures, or holes 74304 that correspond to each protrusion 74230 and are adapted to receive the protrusion 74230 therein when the anvil 74210 is closed to thereby retain and prevent the adjunct 4300 from moving during the cutting and firing procedure.

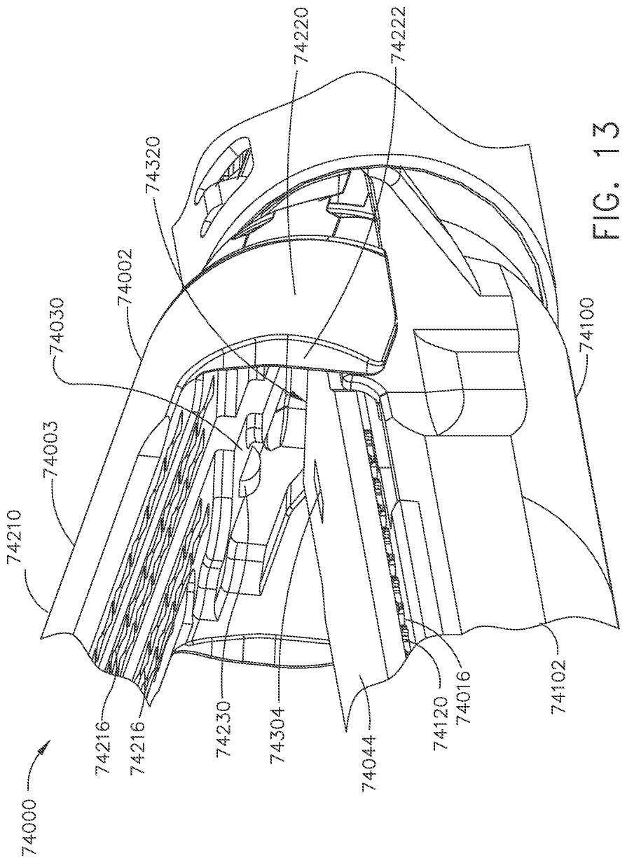

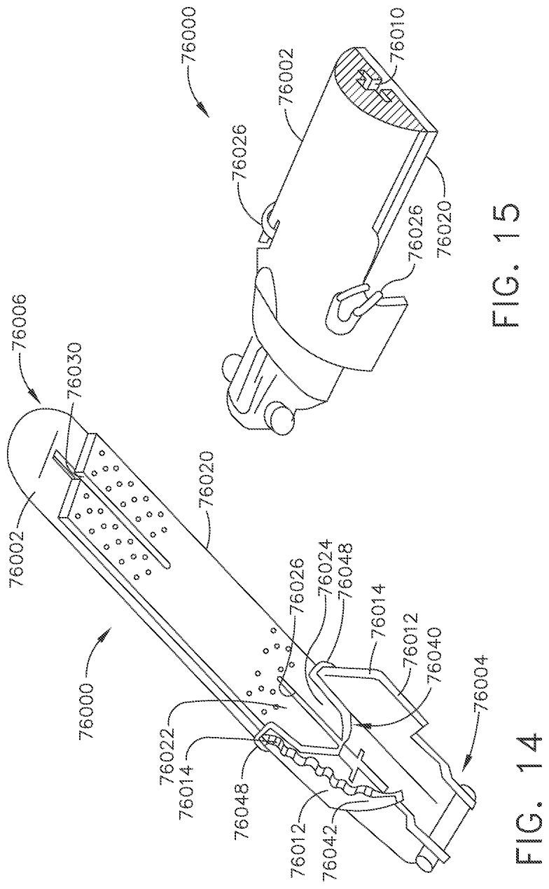

FIGS. 14 and 15 illustrate an anvil 76000 that may be advantageously employed with any of the surgical stapler end effectors disclosed herein. As will be discussed in further detail below, the anvil 76000 is configured to be used with an adjunct 76026. As can be seen in FIGS. 14 and 15, the anvil 76000 comprises an anvil body 76002 that comprises a proximal end portion 76004 and a distal end portion 76006. The anvil body 76002 further comprises a staple-forming undersurface 76008 that extends from the proximal end portion 76004 to the distal end portion 76006 and defines an elongated slot 76010 that is configured to accommodate passage of a firing actuator or sled driver of the surgical stapler. A tissue stop 76012 is formed on each side of the proximal end portion 76004 of the anvil 76000.

The adjunct 76020 may comprise any of the adjuncts disclosed herein with the additional features or attributes discussed below. As shown in FIG. 14, the adjunct 76020 comprises a proximal end 76022 that is configured to extend proximally past the distal edges 76014 of the tissue stops 76012. In the illustrated arrangement, the proximal end 76022 comprises a tapered end portion 76024. Also in the illustrated arrangement, the adjunct 76020 further comprises a proximal slot segment 76026 that extends partially into the adjunct 76020 and coincides with the longitudinal anvil slot 76010. Additionally in the illustrated arrangement, the adjunct 76020 further comprises a distal slot segment 76030 that extends partially inward from a distal end 76028 of the adjunct 76020 and also coincides with the anvil slot 76010. It will be appreciated that the proximal slot segment 76026 and the distal slot segment 76030 facilitates passage of the sled driver or firing actuator of the surgical stapler without contacting the adjunct 76020 during the lengths of the respective slot segments which could otherwise cause undesirable movement of the adjunct 76020 during the cutting and firing process. Other adjuncts may lack one or both of the proximal slot segment 76026 and the distal slot segment 86030.

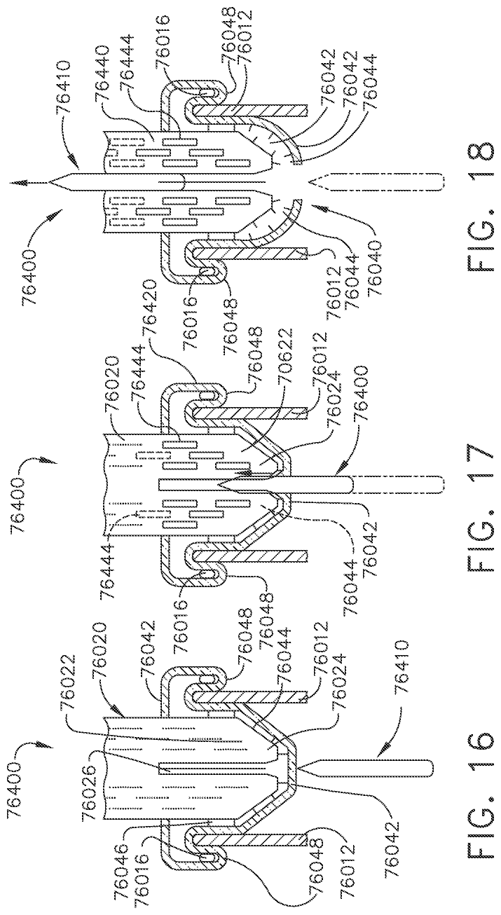

The adjunct 76020 further comprises an adjunct attachment assembly 76040 that is attached to or otherwise formed on the proximal end 76022 of the adjunct 76020 and is configured to releasably engage the proximal end portion 76004 of the anvil 76000. FIGS. 16, 17, and 18 illustrate portions of a surgical stapler end effector 76400 that comprises the anvil 76000 and the adjunct 76020. As shown in FIGS. 16-18, the adjunct attachment assembly 76040 comprises a severable attachment loop 76042 that is releasably attached to the proximal end 76022 of the adjunct 76020. The severable attachment loop 76042 is fabricated from the same material that comprises the adjunct 76020. However, the several attachment loop 76042 may be fabricated from one of the adjunct materials disclosed herein that differs from the adjunct material comprising the adjunct 76020.

As can be seen in FIGS. 14, 16, 17, and 18, the severable attachment loop 76042 is attached to the proximal end portion 76022 by a plurality of tearable links 76044 so that the severable loop 76042 spans across the tapered end portion 76024 and the proximal slot segment 76026 therein. The links 76044 may be formed by forming holes or apertures 76046 in the adjunct material between the links 76044. Additionally, the severable attachment loop comprises a first and second hook or loop portions 76048 formed therein that are configured to hookingly engage a corresponding slotted portion, protrusion, detent, hook or retention feature 76016 formed on each tissue stop 76012 or other portions of the proximal end portion 76004 of the anvil 76000.

FIG. 16 illustrates the position of the adjunct 76020 prior to commencing the cutting and firing process. As can be seen in FIG. 16, the knife 76410 of a sled driver or firing actuator of the surgical stapler is positioned proximal to the severable attachment loop 76042 on the adjunct 76020. FIG. 17 illustrates the position of the proximal end portion 76022 of the adjunct 76020 relative to the proximal-most staple cavities 76444 in a staple cartridge 76440 supported in the end effector 76400. As can be seen in FIG. 17, the proximal end portion 76022 and, more particularly, at least a portion of the tapered end portion 76024 of the adjunct 76020 is proximal to the proximal-most staple cavities 76444.

FIG. 17 illustrates commencement of the cutting and firing process. As can be seen in FIG. 17, the knife has severed the severable attachment loop 76042 as the knife 76410 is advanced distally through the end effector 76400. FIG. 18 illustrates the further advancement of the knife 76410 distally and some of the links 76042 being broken. Once the firing and cutting process has been completed and the adjunct 76020 has been severed and stapled to the severed target tissue that was clamped in the end effector 76400, the end effector jaws can be opened to release the target tissue and adjunct 76020 from the end effector. As the clinician removes the end effector from the stapled target tissue and adjunct, the remaining tearable links 76042 will break to release the adjunct attachment assembly 76040 from the adjunct 76020. Thus, the adjunct attachment assembly 76040 will be withdrawn from the patient with the end effector. Severable hooks may be formed in the adjunct material and may be configured to otherwise releasably engage the hooks or protrusions on the anvil 76000. Such hooks would get torn or severed when the end effector is withdrawn from the stapled target tissue and adjunct to release the adjunct from the anvil leaving the remnants of the adjunct hooks with the staple pieces of adjunct to absorbed in the patient's body. In either cases, those of ordinary skill in the art will appreciate that the adjunct attachment assembly and hook arrangements serve to prevent the adjunct material from being longitudinally and laterally displaced as the knife pass through the adjunct material.

In addition to any of the adjunct retention arrangements and configurations disclosed herein, a temporary form of pressure sensitive adhesive 75000 may be applied to the device-contacting surface of the adjunct. Pressure sensitive adhesives may be characterized by one of more of the following properties: (1) aggressive and permanent tack; (2) adherence with no more than finger pressure; (3) sufficient ability to hold onto an adherend; and (4) sufficient cohesive strength to be removed cleanly from the adherend. These temporary pressure sensitive adhesives may be advantageously applied to certain portions of each adjunct to further enhance the adhesion of the adjunct to the anvil or staple cartridge to thereby prevent the adjunct from slipping, moving or bunching during firing. For example, these pressure sensitive adhesives may be applied only to a proximal portion of the device-contacting surface and/or they may be applied along the length of the adjuncts as well. Examples of such adhesives 75000 include, but are not limited to: poly vinyl pyrrolidone (PVP), poly vinyl acetate (PVA), acrylic based adhesives, and cyanoacrylate adhesives, among others. A more aggressive (stickier) form of adhesive 75000 may be applied only to the device-contacting surface of the adjunct that protrudes proximally beyond the distal edges of the tissue stops. For example, a small amount of PVP based adhesive may be applied to the device-contacting surface that is adjacent to the proximal end of the adjunct (and proximal to the distal edges of the tissue stops) to further enhance the retention of the proximal end of the adjunct to the device (anvil or staple cartridge). The properties of the adhesive may be further tuned to increase the adhesive's resistance to moisture intrusion/softening by reducing the amount of plasticizer added to the adhesive. Some adhesives such as PVP, however, become lubricious when wet making the portions of the device to which it is applied difficult to dry between uses. Thus, in at least some applications, it may be desirable to avoid applying those adhesives to the portions of the anvil that are proximal to the tissue stop distal edges.

FIG. 2 illustrates an amount of temporary pressure-sensitive adhesive 75000 that can be applied to a proximal end portion of the first device-contacting surface 70064. FIG. 4 illustrates an amount of temporary pressure-sensitive adhesive 75000 that can be applied to a proximal end portion of the first device-contacting surface 70074′.

Various aspects of the subject matter described herein are set out in the following examples.

Example 1—An adjunct (70050′, 71050, 71050′, 72050, 74300, 76020) for use with a surgical stapler (70000). The adjunct comprises a first layer (70060) comprising a first material (70062), and a second film layer (70070′) attached to the first layer and defining a continuous textured device-contacting surface (70074′, 70078) configured to establish a frictional interface with a corresponding portion of the surgical stapler. The second film layer comprises a second material (70072) that differs from the first material. The adjunct comprises a third film layer (70080) attached to the first layer and defining a tissue-contacting surface (70082).

Example 2—The adjunct (70050′) of Example 1, wherein the continuous textured device-contacting surface (70074′) is planar.

Example 3—The adjunct (70050′, 71050′) of Examples 1 or 2, wherein the continuous textured device-contacting surface (70074′, 70078) is configured to establish a frictional interface with a staple-forming undersurface (70032) of an anvil (70030) of the surgical stapler.

Example 4—The adjunct (71050′) of Examples 1, 2, or 3, wherein the continuous textured device-contacting surface (70078) comprises a plurality of contoured features (70076), wherein each contoured feature corresponds to a staple-forming pocket (70034) in the staple-forming undersurface (70032) of the anvil (70030).

Example 5—The adjunct (74300, 76020) of Examples 1, 2, 3, or 4, further comprising at least one anvil attachment feature (74222, 76040) configured to releasably engage a corresponding adjunct retainer (74230, 76016) on an anvil (74210, 76400).

Example 6—The adjunct (74300) of Example 5, wherein the at least one anvil attachment feature (74222) comprises an aperture through a proximal end of the adjunct (74300) configured to receive a corresponding one of the adjunct retainers (74230) on the anvil (74210).

Example 7—The adjunct (76020) of Example 5, wherein the at least one anvil attachment feature (76040) comprise a hook (76042) on a proximal end (76022) of the adjunct (76020) that is configured to hookingly engage a corresponding adjunct retainer (76016) on the proximal end (76004) of the anvil (76000).

Example 8—The adjunct (76020) of Example 7, wherein the hook (76042) comprises a first loop (76048) configured to hookingly engage a first hook portion (76016) on the proximal end (76004) of the anvil (76000), a second loop (76048) configured to hookingly engage a second hook portion (76016) on the proximal end (76004) of the anvil (76000), and a severable attachment loop (76042) coupled to the first loop (76048) and the second loop (76048) and extending therebetween, wherein the severable attachment loop (76042) is configured to be severed by a knife (76410) of the surgical stapler and detached from the adjunct (76020).

Example 9—The adjunct (76020) of Example 8, wherein the severable attachment loop (76042) is removably attached to a proximal end (70624) of the adjunct (76020) by a plurality of tearable links (76044).

Example 10—The adjunct of Examples 1, 2, 3, 4, 5, 6, 7, 8, or 9, further comprising an adhesive material (75000) on a proximal portion of the adjunct that extends proximally beyond a distal most portion of tissue stops formed on the anvil.

Example 11—The adjunct of Examples 1 or 2, wherein the continuous textured device-contacting surface (70074′, 70082′) is configured to establish a frictional interface with a deck surface (70028) of a surgical staple cartridge (70020) of the surgical stapler.

Example 12—The adjunct of Example 11, wherein the continuous textured device-contacting surface (70082′) comprises a plurality of concave contoured features (70086), wherein each concave contoured feature corresponds to a pocket extension (70029) protruding from the deck surface (70028) of the surgical staple cartridge (70020).

Example 13—The adjunct of Examples 11 or 12, further comprising at least one cartridge retention feature (74304) configured to releasably engage a corresponding adjunct retainer on the anvil.

Example 14—The adjunct of Example 13, wherein the at least one cartridge retention attachment feature comprises an aperture through a proximal end of the adjunct configured to receive a corresponding one of the adjunct retainers on the anvil when the anvil is in a closed position.

Example 15—The adjunct of Examples 11, 12, 13, or 14, further comprising an adhesive material (75000) on a proximal portion of the adjunct that extends proximally beyond a distal-most portion of tissue stops formed on the anvil.

Example 16—An adjunct for use with a surgical stapler. The adjunct comprises a first layer comprising a first material, a second film layer attached to the first layer and defining a continuous textured device-contacting surface configured to establish a frictional interface with a corresponding portion of the surgical stapler. The second film layer comprises a second material that differs from the first material. The adjunct comprises a third film layer attached to the first layer and defining a tissue-contacting surface.

Example 17—The adjunct of Example 16, wherein said continuous textured device-contacting surface is planar.

Example 18—The adjunct of Examples 16 or 17, wherein the continuous textured device-contacting surface is configured to establish a frictional interface with a staple-forming undersurface of an anvil of the surgical stapler.

Example 19—The adjunct of Example 18, wherein the continuous textured device-contacting surface comprises a plurality of contoured features, wherein each contoured feature corresponds to a staple-forming pocket in the staple-forming undersurface of the anvil.

Example 20—The adjunct of Examples 16, 17, 18, or 19, further comprising at least one anvil attachment feature configured to releasably engage a corresponding adjunct retainer on an anvil.

Example 21—The adjunct of Example 20, wherein the at least one anvil attachment feature comprises an aperture through a proximal end of the adjunct configured to receive a corresponding one of the adjunct retainers on the anvil.

Example 22—The adjunct of Example 20, wherein the at least one anvil attachment feature comprise a hook on a proximal end of the adjunct that is configured to hookingly engage a corresponding adjunct retainer on the proximal end of the anvil.

Example 23—The adjunct of Example 22, wherein the hook comprises a first hook configured to hookingly engage a first hook portion on the proximal end of the anvil, a second hook configured to hookingly engage a second hook portion on the proximal end of the anvil and a severable attachment loop coupled to the first hook and the second hook and extending therebetween, wherein the severable attachment loop is configured to be severed by a knife of the surgical stapler and detached from the adjunct.

Example 24—The adjunct of Example 23, wherein the severable attachment loop is removably attached to a proximal end of the adjunct by a plurality of tearable links.

Example 25—The adjunct of Examples 16, 17, 18, 19, 20, 21, 22, 23, or 24, further comprising an adhesive material on a proximal portion of the adjunct that extends proximally beyond a distal most portion of tissue stops formed on the anvil.

Example 26—The adjunct of Examples 16, 17, 18, 19, 20, 21, 22, 23, or 24, wherein the continuous textured device-contacting surface is configured to establish a frictional interface with a deck surface of a surgical staple cartridge of the surgical stapler.

Example 27—The adjunct of Example 26, wherein the continuous textured device-contacting surface comprises a plurality of concave contoured features, wherein each concave contoured feature corresponds to a pocket extension protruding from the deck surface of the surgical staple cartridge.

Example 28—The adjunct of Example 26, further comprising at least one cartridge retention feature configured to releasably engage a corresponding adjunct retainer on the anvil.

Example 29—The adjunct of Example 28, wherein the at least one cartridge retention attachment feature comprises an aperture through a proximal end of the adjunct configured to receive a corresponding one of the adjunct retainers on the anvil when the anvil is in a closed position.

Example 30—A surgical stapler end effector. The surgical stapler end effector comprises a first jaw, an anvil movably supported relative to the first jaw, wherein the anvil comprises a proximal end portion, a distal end portion, and a staple-forming undersurface extending from the proximal end portion to the distal end portion. The surgical stapler end effector comprises an adjunct removably coupled to the anvil adjacent the staple-forming undersurface, wherein the adjunct comprises an adjunct retention assembly comprising at least one hook formed on a proximal end of the adjunct and configured to releasably engage a corresponding hook portion of the proximal end of the anvil.

Example 31—The surgical stapler end effector of Example 30, wherein the adjunct retention assembly comprises a first loop configured to hookingly engage a first hook portion on the proximal end of the anvil, a second loop configured to hookingly engage a second hook portion on the proximal end of the anvil, and a severable attachment loop coupled to the first loop and the second loop and extending therebetween, wherein the severable attachment loop is configured to be severed by a knife of the surgical stapler end effector and detached from the adjunct.

Example 32—The surgical stapler end effector of Example 31, wherein the severable attachment loop is removably attached to a proximal end of the adjunct by a plurality of tearable links.

Example 33—The surgical stapler end effector of Examples 30, 31, or 32, wherein the anvil further comprises at least one tissue stop that comprises at least one distal tissue stop edge, and wherein the at least one hook of the adjunct retention assembly is proximal to the at least one distal tissue stop edge.

Example 34—A surgical stapler end effector. The surgical stapler end effector comprises a first jaw and an anvil movably supported relative to the first jaw between an open position and a closed position. The anvil comprises a proximal end portion, a distal end portion and an undersurface comprising a staple-forming portion. The anvil comprises at least one tissue stop on the proximal end, wherein each tissue stop defines a distal tissue stop edge, and at least one projection protruding from a portion of the undersurface that is proximal to each of the distal tissue stop edges, wherein each projection is configured to pin a proximal end of an adjunct positioned adjacent the undersurface between the projection and a corresponding portion of the first jaw to prevent movement of the adjunct relative to the first jaw when the anvil is in the closed position.

Example 35—The surgical stapler end effector of Example 34, wherein the first jaw comprises a surgical staple cartridge comprising a cartridge proximal end, a cartridge distal end, a cartridge deck surface extending between the cartridge proximal end and the cartridge distal end; a cavity in the cartridge deck surface corresponding to each of the at least one projections and configured to receive therein a portion of the corresponding projection when the anvil is in the closed position. The surgical stapler end effector further comprises an adjunct comprising an adjunct proximal end, and an aperture therethrough corresponding to each protrusion of the anvil and configured to receive therethrough the corresponding protrusion when the anvil is in the closed position and wherein the adjunct is positioned on the cartridge deck surface.

Many of the surgical instrument systems described herein are motivated by an electric motor; however, the surgical instrument systems described herein can be motivated in any suitable manner. The surgical instrument systems described herein can be motivated by a manually-operated trigger, for example. The motors disclosed herein may comprise a portion or portions of a robotically controlled system. Moreover, any of the end effectors and/or tool assemblies disclosed herein can be utilized with a robotic surgical instrument system. U.S. patent application Ser. No. 13/118,241, entitled SURGICAL STAPLING INSTRUMENTS WITH ROTATABLE STAPLE DEPLOYMENT ARRANGEMENTS, now U.S. Pat. No. 9,072,535, for example, discloses several examples of a robotic surgical instrument system in greater detail.

The surgical instrument systems described herein have been described in connection with the deployment and deformation of staples; however, the present disclosure may not be so limited. The present disclosure envisions that fasteners other than staples can be deployed, such as clamps or tacks, for example. Moreover, the present disclosure envisions utilizing any suitable means for sealing tissue. An end effector in accordance with the present disclosure can comprise electrodes configured to heat and seal the tissue. Also, an end effector in accordance with the present disclosure can apply vibrational energy to seal the tissue.

The entire disclosures of:
U.S. Pat. No. 5,403,312, entitled ELECTROSURGICAL HEMOSTATIC DEVICE, which issued on Apr. 4, 1995;
U.S. Pat. No. 7,000,818, entitled SURGICAL STAPLING INSTRUMENT HAVING SEPARATE DISTINCT CLOSING AND FIRING SYSTEMS, which issued on Feb. 21, 2006;
U.S. Patent No. 7,422, 139, entitled MOTOR-DRIVEN SURGICAL CUTTING AND FASTENING INSTRUMENT WITH TACTILE POSITION FEEDBACK, which issued on Sep. 9, 2008;
U.S. Pat. No. 7,464,849, entitled ELECTRO-MECHANICAL SURGICAL INSTRUMENT WITH CLOSURE SYSTEM AND ANVIL ALIGNMENT COMPONENTS, which issued on Dec. 16, 2008;
U.S. Pat. No. 7,670,334, entitled SURGICAL INSTRUMENT HAVING AN ARTICULATING END EFFECTOR, which issued on Mar. 2, 2010;
U.S. Pat. No. 7,753,245, entitled SURGICAL STAPLING INSTRUMENTS, which issued on Jul. 13, 2010;
U.S. Pat. No. 8,393,514, entitled SELECTIVELY ORIENTABLE IMPLANTABLE FASTENER CARTRIDGE, which issued on Mar. 12, 2013;
U.S. patent application Ser. No. 11/343,803, entitled SURGICAL INSTRUMENT HAVING RECORDING CAPABILITIES, now U.S. Pat. No. 7,845,537;
U.S. patent application Ser. No. 12/031,573, entitled SURGICAL CUTTING AND FASTENING INSTRUMENT HAVING RF ELECTRODES, filed Feb. 14, 2008;
U.S. patent application Ser. No. 12/031,873, entitled END EFFECTORS FOR A SURGICAL CUTTING AND STAPLING INSTRUMENT, filed Feb. 15, 2008, now U.S. Pat. No. 7,980,443;
U.S. patent application Ser. No. 12/235,782, entitled MOTOR-DRIVEN SURGICAL CUTTING INSTRUMENT, now U.S. Pat. No. 8,210,411;
U.S. patent application Ser. No. 12/235,972, entitled MOTORIZED SURGICAL INSTRUMENT, now U.S. Pat. No. 9,050,083.
U.S. patent application Ser. No. 12/249,117, entitled POWERED SURGICAL CUTTING AND STAPLING APPARATUS WITH MANUALLY RETRACTABLE FIRING SYSTEM, now U.S. Pat. No. 8,608,045;
U.S. Patent Application Ser. No. 12/647, 100, entitled MOTOR-DRIVEN SURGICAL CUTTING INSTRUMENT WITH ELECTRIC ACTUATOR DIRECTIONAL CONTROL ASSEMBLY, filed Dec. 24, 2009, now U.S. Pat. No. 8,220,688;
U.S. patent application Ser. No. 12/893,461, entitled STAPLE CARTRIDGE, filed Sep. 29, 2012, now U.S. Pat. No. 8,733,613;
U.S. patent application Ser. No. 13/036,647, entitled SURGICAL STAPLING INSTRUMENT, filed Feb. 28, 2011, now U.S. Pat. No. 8,561,870;
U.S. patent application Ser. No. 13/118,241, entitled SURGICAL STAPLING INSTRUMENTS WITH ROTATABLE STAPLE DEPLOYMENT ARRANGEMENTS, now U.S. Pat. No. 9,072,535;
U.S. patent application Ser. No. 13/524,049, entitled ARTICULATABLE SURGICAL INSTRUMENT COMPRISING A FIRING DRIVE, filed on Jun. 15, 2012, now U.S. Pat. No. 9,101,358;
U.S. patent application Ser. No. 13/800,025, entitled STAPLE CARTRIDGE TISSUE THICKNESS SENSOR SYSTEM, filed on Mar. 13, 2013, now U.S. Pat. No. 9,345,481;
U.S. patent application Ser. No. 13/800,067, entitled STAPLE CARTRIDGE TISSUE THICKNESS SENSOR SYSTEM, filed on Mar. 13, 2013, now U.S. Patent Application Publication No. 2014/0263552;
U.S. Patent Application Publication No. 2007/0175955, entitled SURGICAL CUTTING AND FASTENING INSTRUMENT WITH CLOSURE TRIGGER LOCKING MECHANISM, filed Jan. 31, 2006; and
U.S. Patent Application Publication No. 2010/0264194, entitled SURGICAL STAPLING INSTRUMENT WITH AN ARTICULATABLE END EFFECTOR, filed Apr. 22, 2010, now U.S. Pat. No. 8,308,040, are hereby incorporated by reference herein.

Although various devices have been described herein in connection with certain embodiments, modifications and variations to those embodiments may be implemented. Particular features, structures, or characteristics may be combined in any suitable manner in one or more embodiments. Thus, the particular features, structures, or characteristics illustrated or described in connection with one embodiment may be combined in whole or in part, with the features, structures or characteristics of one ore more other embodiments without limitation. Also, where materials are disclosed for certain components, other materials may be used. Furthermore, according to various embodiments, a single component may be replaced by multiple components, and multiple components may be replaced by a single component, to perform a given function or functions. The foregoing description and following claims are intended to cover all such modification and variations.

The devices disclosed herein can be designed to be disposed of after a single use, or they can be designed to be used multiple times. In either case, however, a device can be reconditioned for reuse after at least one use. Reconditioning can include any combination of the steps including, but not limited to, the disassembly of the device, followed by cleaning or replacement of particular pieces of the device, and subsequent reassembly of the device. In particular, a reconditioning facility and/or surgical team can disassemble a device and, after cleaning and/or replacing particular parts of the device, the device can be reassembled for subsequent use. Those skilled in the art will appreciate that reconditioning of a device can utilize a variety of techniques for disassembly, cleaning/replacement, and reassembly. Use of such techniques, and the resulting reconditioned device, are all within the scope of the present application.

The devices disclosed herein may be processed before surgery. First, a new or used instrument may be obtained and, when necessary, cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, and/or high-energy electrons. The radiation may kill bacteria on the instrument and in the container. The sterilized instrument may then be stored in the sterile container. The sealed container may keep the instrument sterile until it is opened in a medical facility. A device may also be sterilized using any other technique known in the art, including but not limited to beta radiation, gamma radiation, ethylene oxide, plasma peroxide, and/or steam.

It is worthy to note that any reference numbers included in the appended claims are used to reference exemplary embodiments/elements described in the present disclosure. Accordingly, any such reference numbers are not meant to limit the scope of the subject matter recited in the appended claims.

While this invention has been described as having exemplary designs, the present invention may be further modified within the spirit and scope of the disclosure. This application is therefore intended to cover any variations, uses, or adaptations of the invention using its general principles.

1-15. (canceled)

16. An adjunct for use with a surgical stapler, wherein said adjunct comprises:
a first layer comprising a first material;
a second film layer attached to the first layer and defining a continuous textured device-contacting surface configured to establish a frictional interface with a corresponding portion of the surgical stapler, wherein the second film layer comprises a second material that differs from the first material; and
a third film layer attached to the first layer and defining a tissue-contacting surface.

17. The adjunct of claim 16, wherein said continuous textured device-contacting surface is planar.

18. The adjunct of claim 16, wherein the continuous textured device-contacting surface is configured to establish a frictional interface with a staple-forming undersurface of an anvil of the surgical stapler.

19. The adjunct of claim 18, wherein the continuous textured device-contacting surface comprises a plurality of contoured features, wherein each contoured feature corresponds to a staple-forming pocket in the staple-forming undersurface of the anvil.

20. The adjunct of claim 16, further comprising at least one anvil attachment feature configured to releasably engage a corresponding adjunct retainer on an anvil.

21. The adjunct of claim 20, wherein the at least one anvil attachment feature comprises an aperture through a proximal end of the adjunct configured to receive a corresponding one of the adjunct retainers on the anvil.

22. The adjunct of claim 20, wherein the at least one anvil attachment feature comprise a hook on a proximal end of the adjunct that is configured to hookingly engage a corresponding adjunct retainer on the proximal end of the anvil.

23. The adjunct of claim 22, wherein the hook comprises:
a first hook configured to hookingly engage a first hook portion on the proximal end of the anvil;
a second hook configured to hookingly engage a second hook portion on the proximal end of the anvil;
a severable attachment loop coupled to the first hook and the second hook and extending therebetween, wherein the severable attachment loop is configured to be severed by a knife of the surgical stapler and detached from the adjunct.

24. The adjunct of claim 23, wherein the severable attachment loop is removably attached to a proximal end of the adjunct by a plurality of tearable links.

25. The adjunct of claim 16, further comprising an adhesive material on a proximal portion of the adjunct that extends proximally beyond a distal most portion of tissue stops formed on the anvil.

26. The adjunct of claim 16, wherein the continuous textured device-contacting surface is configured to establish a frictional interface with a deck surface of a surgical staple cartridge of the surgical stapler.

27. The adjunct of claim 26, wherein the continuous textured device-contacting surface comprises a plurality of concave contoured features, wherein each concave contoured feature corresponds to a pocket extension protruding from the deck surface of the surgical staple cartridge.

28. The adjunct of claim 26, further comprising at least one cartridge retention feature configured to releasably engage a corresponding adjunct retainer on the anvil.

29. The adjunct of claim 28, wherein the at least one cartridge retention attachment feature comprises an aperture through a proximal end of the adjunct configured to receive a corresponding one of the adjunct retainers on the anvil when the anvil is in a closed position.

30. A surgical stapler end effector, comprising:
a first jaw;
an anvil movably supported relative to the first jaw, wherein the anvil comprises a proximal end portion, a distal end portion, and a staple-forming undersurface extending from the proximal end portion to the distal end portion; and
an adjunct removably coupled to the anvil adjacent the staple-forming undersurface, wherein the adjunct comprises an adjunct retention assembly comprising at least one hook formed on a proximal end of the adjunct and configured to releasably engage a corresponding hook portion of the proximal end of the anvil.

31. The surgical stapler end effector of claim 30, wherein the adjunct retention assembly comprises:
a first loop configured to hookingly engage a first hook portion on the proximal end of the anvil;
a second loop configured to hookingly engage a second hook portion on the proximal end of the anvil;
a severable attachment loop coupled to the first loop and the second loop and extending therebetween, wherein the severable attachment loop is configured to be severed by a knife of the surgical stapler end effector and detached from the adjunct.

32. The surgical stapler end effector of claim 31, wherein the severable attachment loop is removably attached to a proximal end of the adjunct by a plurality of tearable links.

33. The surgical stapler end effector of claim 30, wherein the anvil further comprises at least one tissue stop that comprises at least one distal tissue stop edge, and wherein the at least one hook of the adjunct retention assembly is proximal to the at least one distal tissue stop edge.

34. A surgical stapler end effector, comprising:
a first jaw; and
an anvil movably supported relative to the first jaw between an open position and a closed position, wherein the anvil comprises:
a proximal end portion;
a distal end portion;
an undersurface comprising a staple-forming portion;
at least one tissue stop on the proximal end, wherein each tissue stop defines a distal tissue stop edge; and
at least one projection protruding from a portion of the undersurface that is proximal to each of the distal tissue stop edges, wherein each projection is configured to pin a proximal end of an adjunct positioned adjacent the undersurface between the projection and a corresponding portion of the first jaw to prevent movement of the adjunct relative to the first jaw when the anvil is in the closed position.

35. The surgical stapler end effector of claim 34, wherein the first jaw comprises a surgical staple cartridge comprising:
a cartridge proximal end;
a cartridge distal end;
a cartridge deck surface extending between the cartridge proximal end and the cartridge distal end;
a cavity in the cartridge deck surface corresponding to each of the at least one projections and configured to receive therein a portion of the corresponding projection when the anvil is in the closed position, and wherein the surgical stapler end effector further comprises an adjunct comprising:
an adjunct proximal end; and
an aperture therethrough corresponding to each protrusion of the anvil and configured to receive therethrough the corresponding protrusion when the anvil is in the closed position and wherein the adjunct is positioned on the cartridge deck surface.
